(19) United States
(12) Patent Application Publication
(10) Pub. No.: US 2016/0089508 A1
(43) Pub. Date: Mar. 31, 2016

(54) VAPOR INHALATION DEVICE

(71) Applicant: ALTR, Inc., New York, NY (US)

(72) Inventors: Caleb Smith, Brooklyn, NY (US); Adel Dahdal, Stockholm (SE); Thomas Hallaran, New York, NY (US)

(21) Appl. No.: 14/496,381

(22) Filed: Sep. 25, 2014

(51) Int. Cl. A61M 15/06; A61M 15/00; A24F 47/00

(52) U.S. Cl. CPC A61M 15/06; A24F 47/008; A61M 15/0085; A61M 15/0003; A61M 15/009; A61M 2205/3584; A61M 2205/123; A61M 2202/049; A61M 2205/3379; A61M 2205/50; A61M 2205/3673; A61M 2205/3334; A61M 2205/3344; A61M 2205/8212; A61M 2205/127

(57) ABSTRACT

A vapor inhalation device (10) comprising:

a housing (12) adapted to enable a user to inhale vapor, simulating the effects of smoking, the housing being adapted to receive a cartridge (14) for containing liquids;
the cartridge (14) comprising or connected to a first release device (15a) arranged to vaporize and release a first liquid (A) comprising a first substance (a) from a first reservoir (16a) into the housing (12); and to a second release device (15b) arranged to vaporize and release a second liquid (B) from a second reservoir (16b), wherein the housing (12) is adapted to mix the vaporized first (A) and second liquids (B) such that the user can inhale vapor containing the first substance (a) of a particular amount;
a communication unit (17) configured to receive and transmit data for controlling the inhalation device (10);
a controller (18) configured to communicate with the communication unit (17) and to receive data from the communication unit; and to
determine an amount of first substance (a) to be released based on the data; and
control the first and second release devices (15a, 15b) based on the determined amount of first substance (a).

### TECHNICAL FIELD

The present disclosure relates generally to the field of vapor inhalation devices and more particularly to control mechanisms for electric vapor inhalation devices.

### BACKGROUND

Electric vapor inhalation devices, commonly known as e-cigarettes, can be used to simulate a cigarette or a cigar. For example, an electric vapor inhalation device can vaporize a liquid including a drug such as nicotine. The vapor inhalation device uses electricity from a battery to heat a resistance coil, a so-called “atomizer”, which when activated heats the liquid to produce vapor. A user of the vapor inhalation device can inhale the vapor and have an experience similar to smoking a traditional combustible cigarette or cigar.

### SUMMARY

According to an illustrative embodiment of the disclosure, there is provided a vapor inhalation device comprising a housing adapted to enable a user to inhale vapor, simulating the effects of smoking. The housing is adapted to receive a cartridge for containing liquids, comprising or connected to a first release device arranged to vaporize and release a first liquid in a first reservoir comprising a first substance into the housing and to a second release device arranged to vaporize and release a second liquid in a second reservoir, wherein the housing is adapted to mix the vaporized first and second liquids such that the user can inhale vapor containing the first substance of a particular amount. The inhalation device further comprises a communication unit, which is configured to receive and transmit data for controlling the inhalation device. A controller is provided and configured to communicate with the communication unit and to receive data from the communication unit; and to determine an amount of first substance to be released based on the data; and control the first and second release devices based on the determined amount of the first substance. Typically, the first substance is a drug substance such as nicotine.

The at least two release devices can be heaters or atomizers adapted to work with the cartridge containing at least two reservoirs containing liquids, a power source such as a battery, and the controller. Typically, the device uses electricity from a battery to heat a resistance coil, typically embodied as a so-called “atomizer”, which when activated heats the liquid to produce vapor. The release devices and the cartridge can alternatively be integrated with each other to an exchangeable unit, which can be inserted into or removed from the housing. The controller controls the release devices to deliver a predetermined amount of the substance, typically a drug such as nicotine, to the user. The inhalation device can be a long cylindrical shape similar to a cigarette or cigar.

According to an illustrative embodiment of the disclosure, the communication unit is configured to communicate with and be controlled by a mobile communication device such as a smart-phone having an application program product configured to control the inhalation device.

According to an illustrative embodiment of the disclosure, the controller is configured to control the first and second release devices to deliver the first substance in precise increments, which can be set by the user, in an amount of 0 to 100% concentration of the first substance.

According to an illustrative embodiment of the disclosure, the vapor inhalation device further comprises a third release device, wherein the first release device vaporizes the first liquid being a solvent and the first substance in the first reservoir, the second release device vaporizes the first liquid only in the second reservoir and the third release device vaporizes the first liquid and a placebo substance in a third reservoir. Only two reservoirs are required in accordance with the first described embodiment, wherein a placebo fluid may be mixed with a non-nicotine fluid, but any other higher number of reservoirs than three is also possible. According to alternative embodiments of the disclosure, the release devices and/or the reservoirs can be arranged in parallel or alternatively in series to each other. Herein, the terms “parallel” and “series” means the direction of airflow.

According to an illustrative embodiment of the disclosure, the inhalation device can also receive and transmit data related to settings and usage. Advantageously, the inhalation device can also monitor an amount of substance delivered to a user.

Advantageously, the inhalation device can control and/or limit amount of substance delivered to help the user limit the consumption of a certain substance such as nicotine.

Advantageously, the inhalation device can also control and/or limit amount of substance delivered such that a user can define a limit on an intake amount of the substance per use or for a certain period by the user.

According to another illustrative embodiment of the disclosure, there is provided a cartridge for an inhalation device as described above. The cartridge comprises or is connectable to a first release device arranged to vaporize and release a first liquid comprising a first substance from a first reservoir of the cartridge and to a second release device arranged to vaporize and release a second liquid from a second reservoir of the cartridge into the housing.

According to another illustrative embodiment of the disclosure, the cartridge comprises or is connectable to a third release device arranged to vaporize liquid from a third reservoir, wherein the first reservoir contains the first liquid being a solvent and the first substance, the second reservoir contains the first liquid only and the third reservoir contains the first liquid and a placebo substance.

Another illustrative embodiment of the disclosure is related to a non-transitory computer-readable medium having instructions stored thereon that, if executed by a computing device, cause the computing device to perform operations for controlling an inhalation device.

The purpose of the vapor inhalation device and other apparatus described in this disclosure is to deliver a precise user defined amount of substance a, such as a drug, in particular nicotine. The vapor inhalation is communicating with and controlled by the user's mobile communication device

### BRIEF DESCRIPTION OF DRAWING FIGURES

The foregoing and other features of the present disclosure will become more fully apparent from the following description and appended claims, taken in conjunction with the accompanying drawings. Understanding that these drawings depict only several embodiments in accordance with the disclosure and are; therefore, not to be considered limiting of its scope, the disclosure will be described with additional specificity and detail through use of the accompanying drawings.

FIG. 1 is a section view of a vapor inhalation in accordance with an illustrative embodiment of the disclosure;

FIG. 2a is a schematic view of a cartridge having reservoirs arranged in parallel to each other in accordance with an illustrative embodiment of the disclosure;

FIG. 2b is a schematic view of a cartridge having reservoirs arranged in series to each other in accordance with another illustrative embodiment of the disclosure; FIG. 2c is a schematic view of a cartridge having reservoirs arranged in series to each other in accordance with another illustrative embodiment of the disclosure; and

FIG. 3 is a schematic detail view in section of one of the “cartomizers” of the cartridges illustrated in FIG. 2a-c; and FIG. 4 is a flow-chart of a method performed in the controller according to an embodiment of the disclosure.

### DETAILED DESCRIPTION OF EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

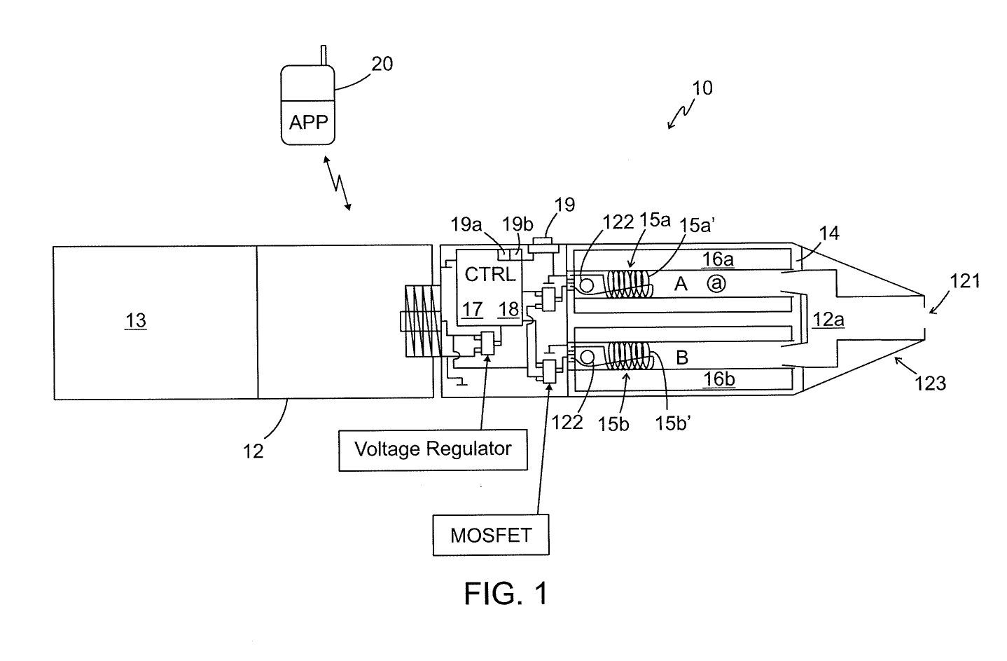

Referring to FIG. 1, a section view of a vapor inhalation device 10 in accordance with an illustrative embodiment is illustrated.

The inhalation device 10 comprises a housing 12, a cartridge 14 for containing liquids, a power source 13, a communication unit 17, a controller 18, typically a so-called “micro-controller”, and possibly a light device (not shown) for producing a glowing light similar to a lighted cigarette.

The housing 12 adapted to enable a user to inhale vapor and/or smoke, simulating the effects of smoking has a long cylindrical shape, for example, the shape and size of a cigar, cigarillo, or cigarette. In other embodiments, the housing 12 can be embodied as other smoking or vapor delivery articles such as a hookah, a pipe, an inhaler, or a humidifier. The housing 12 includes an outlet hole 121, typically at a first end (i.e., a mouth end) but also other locations are possible, where a user can place his lips to breathe in vapor or gas generated by the inhalation device 10. The housing 12 can include one or more, herein two inlet holes 122 to allow air to enter the housing 12 when a user sucks on the first end 123, wherein the outlet hole 121 is located. The housing 12 can be made of paper (e.g., paper rolled up in tubular shape), plastic, metal, wood, glass, or any other material.

The cartridge 14 comprises (as illustrated in this drawing FIG. 1) or is connected to (not illustrated in this drawing FIG. 1) a first release device 15a arranged to vaporize and release a first liquid A comprising a first substance a, such as a drug, and in particular a nicotine-containing fluid, from a first reservoir 16a into the housing 12. The cartridge 14 further comprises a second release device 15b arranged to vaporize and release a second liquid B, such as a non-drug, in particular a non-nicotine, containing fluid, from a second reservoir 16b. The housing 12, for instance having a fluid mixing part 12a is adapted to mix the vaporized first A and second liquids B such that the user can inhale vapor containing the first substance a of a particular amount when he/she sucks on the first end 123, on the outlet hole 121.

The vapor inhalation device 10 uses electricity from the power source 13 to heat a resistance element of each of the release devices 15a, 15b, which when activated heats the liquids A, B to produce vapor. The user inhales the vaporized liquid, simulating the effects of smoking a cigarette via the outlet hole 121. Typically the vaporized liquid contains a mix of propylene glycol, vegetable glycol, a drug-substance such as nicotine and a flavor substance, for instance pepper. The vapor inhalation device has no feedback and is open loop, where a pre-determined amount of the drug-substance is set by an operator not being a user or by the user and a ratio of two or more release devices is selected to provide the pre-determined or set amount of the drug. In this way, a high accuracy of drug delivery can be achieved.

The at least two release devices 15a, 15b can be heaters adapted to work with the cartridge containing the at least two reservoirs 16a, 16b containing liquids A, B, the power source 13 and the controller 18, which controls the release devices 15a, 15b. The release devices 15a, 15b and the cartridge 14 can alternatively be integrated with each other to an exchangeable unit, which can be inserted into or removed from the housing 12 (Herein this drawing figure FIG. 1 illustrated inserted). Typically, the first and second release devices 15a, 15b comprises at least one of a heating element, a vibration generator an ultrasonic transducer, a piezoelectric transducer, and an atomizer.

In the embodiment of the disclosure illustrated in FIG. 1, the first and second release devices 15a, 15b comprise atomizers, each comprising a vaporizing coil 15a″, 15b″ that can be heated by electric current I provided by the battery 13. The precise level of substance a, such as a drug, and in particular nicotine is achieved by independently heating the separate release devices, herein the atomizers. Each atomizer 15a, 15b is heated individually by the electric current I, which is controlled by the controller 18. The release devices can be arranged to release the substances simultaneously or serially. This will be explained in more detail below, but first alternative configurations of the reservoirs and the release devices according to different embodiments will be given.

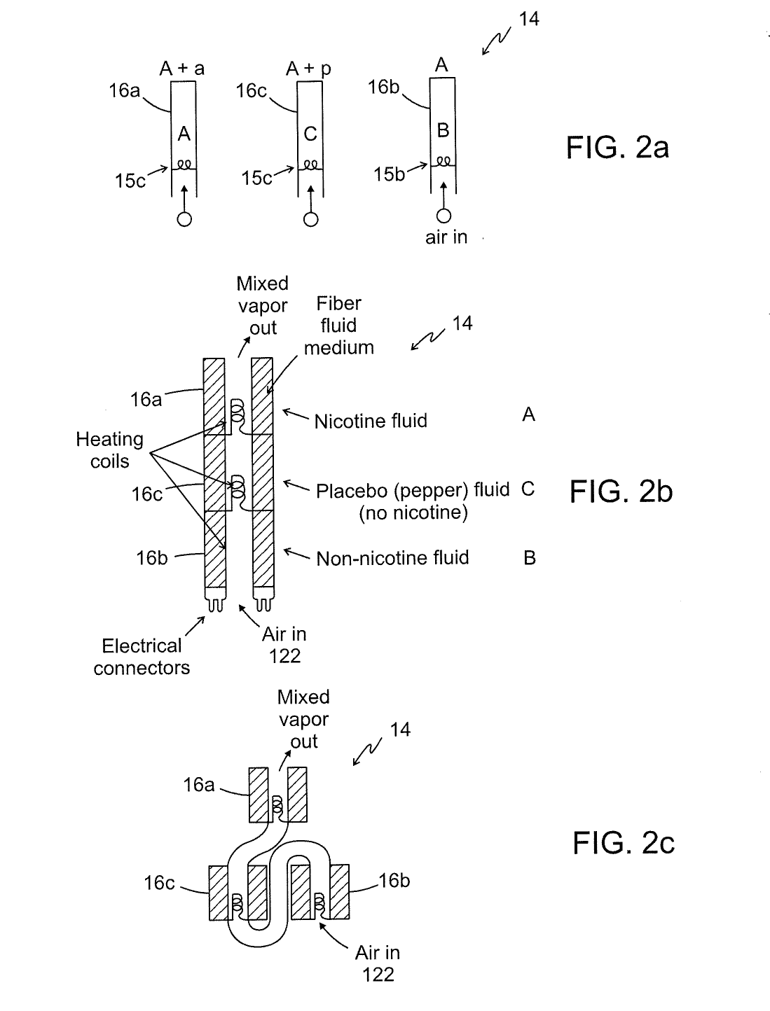

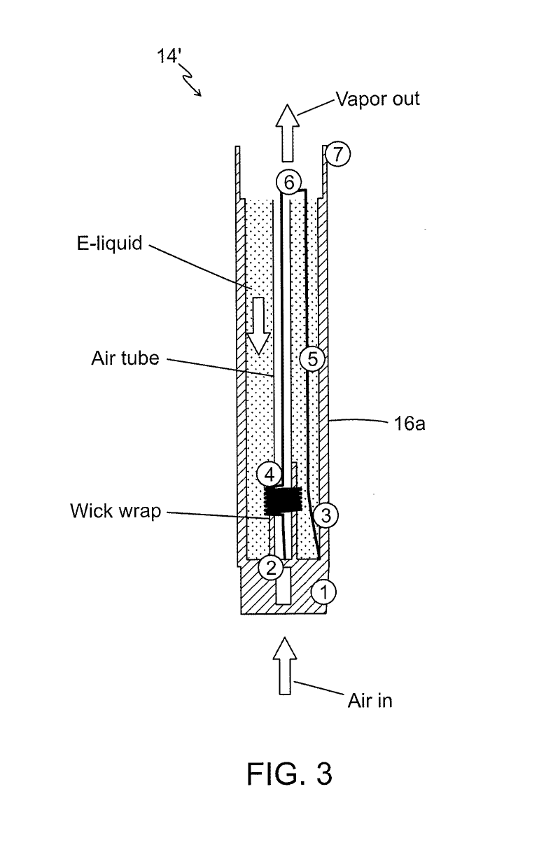

Now is referred to FIG. 2a-c.

FIG. 2a is a schematic view of a cartridge having three reservoirs and release devices arranged in parallel to each other in accordance with an illustrative embodiment of the disclosure.

FIG. 2b is a schematic view of a cartridge having three reservoirs and release devices arranged in series to each other in accordance with another illustrative embodiment of the disclosure. FIG. 2c is a schematic view of a cartridge having three reservoirs and release devices arranged in series to each other in accordance with another illustrative embodiment of the disclosure.

The cartridge 14 illustrated in FIG. 2a is similar to the one illustrated and in relation to FIG. 1, but has three reservoirs 16a, 16b, 16c, and release devices 15a, 15b, 15c instead of two only as the cartridge 14 illustrated and described in relation to in FIG. 1. The cartridge 14 illustrated in FIG. 2a has three separate channels, or in other words, has three “channels” instead of two (channels repeats), which converge at the fluid mixing part 12a where the user inhales the vapor. Typically, therefore, there are three air inlet holes 122 instead of only two as illustrated in relation to FIG. 1. The first reservoir 16a contains the first liquid A being a solvent and the first substance a, the second reservoir 16b contains the first liquid A only and the third reservoir 16c contains a third liquid C containing the first liquid A and a placebo substance p such as pepper, which is different from the embodiment in FIG. 1 having two channels, wherein the placebo fluid p may be mixed with the non-nicotine fluid B. Then in the fluid mixing part 12a, the vapors of the liquids A, B and C are mixed.

As illustrated in FIG. 2b, the three reservoirs 16a, 16b, 16c, and release devices 15a, 15b, 15c are arranged in series. Typically, therefore, there can be only one air inlet hole 122 instead of only three as illustrated in relation to FIG. 2a. The first reservoir 16a contains the first liquid A being a solvent and the first substance a, the second reservoir 16b contains the first liquid A only and the third reservoir 16c contains the first liquid A and the placebo substance p. In the embodiment illustrated in FIG. 2c, the three reservoirs 16a, 16b, 16c, and release devices 15a, 15b, 15c are arranged in series as in FIG. 2b but in another way. The containers can be arranged in any suitable way, suitable for the design and this is only an example. The first reservoir 16a contains the first liquid A being a solvent and the first substance a, the second reservoir 16b contains the first liquid A only and the third reservoir 16c contains the first liquid A and the placebo substance p. Without departing from the invention, any number of channels, i.e. reservoirs is possible.

An advantage with the embodiment of the disclosure compared to prior art device is that the use of different channels provides a very accurate dosage of the first substance a in the vapor.

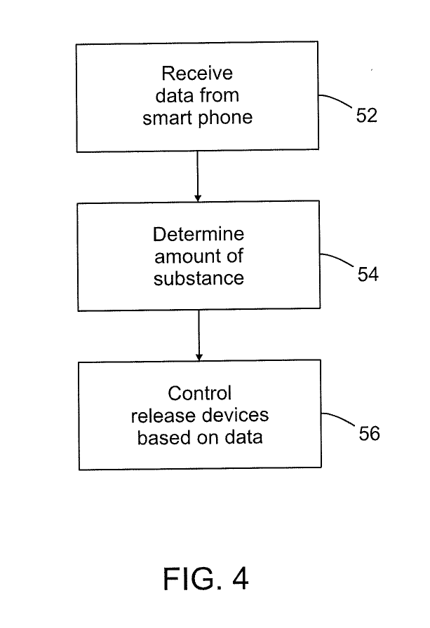

Now is referred to FIG. 3 illustrating a so-called “cartomizer” 14′ according to an example, of which two or three are used in the embodiments illustrated and described in relation to FIG. 1 (two) and FIG. 2a-c (three).

The liquid, which may be the liquid A, B or C as disclosed in FIG. 1 or FIG. 2a-c is contained in a container, herein the first container 16a. A battery connector 1 is connected for power a heater coil 4. A positive electric heater connection 2 and a negative electric heater connection is provided and connected to the heater coil 4. The liquid is contained in a synthetic filler material 5 (illustrated by a grey-shadow) and an air passage is provided. An air-flow is illustrated for a better understanding of the production of vapor.

Now is referred back to FIG. 1.

Other circuitry that may be provided including voltage regulator, field effect transistors MOSFET are only schematically illustrated and labelled but they, or their function, will not be described in more detail, since they are well known for the skilled person to design. The voltage regulator, denoted “Voltage Regulator”, provides pulses to the release devices. The pulses can be provided as regulated voltage or analogue pulses or simulated analogue pulses, or digital pulses for instance, but also in other ways that are suitable. Also an activation button 19 for powering on/off the device is only schematically illustrated, but not described in more detail.

A communication unit 17 is also provided in the vapor inhalation device 10 and configured to receive and transmit data for controlling the vapor inhalation device 10. The communication unit 17 is configured to communicate with and be controlled by a mobile communication device 20 such as a smart-phone having an application program product APP configured to control the inhalation device 10. The communication unit 17 can also be configured to transmit data including at least one of battery charge percentage, or number of individual atomizer activations. The communication unit 17 can be embodied as a BLE SoC Module using communication means such as Bluetooth for communication, but also any other means of similar communication including radio, light or the like is also possible. Using the mobile communication device 20 and downloading the APP is also not described since this is well known for the skilled person. However, the functionality of the application program product APP will be described as follows.

The controller 18 is configured to communicate with the communication unit 17 and to receive data from the communication unit 17 and to determine the amount of the first substance to be released based on the data, and control the first and second release devices 15a, 15b (and possibly the third release device 15c) based on the determined amount of the first substance. According to an embodiment of the disclosure, the controller 18 is configured to control the release devices 15a, 15b, 15c to deliver the first substance an in precise increments, which can be set by the user via the mobile communication device 20, in an amount of 0 to 100% concentration of the first substance a.

The communication unit 17 and the controller 18 can be combined in one module as illustrated in FIG. 1.

Now is referred also to FIG. 4 illustrating a method performed in the controller 18 according to an embodiment of the disclosure.

According to an embodiment of the discloser, the controller 18 comprises a computing device 19a and non-transitory computer-readable medium 19b, that has instructions stored thereon that, if executed by the computing device 19a, cause the computing device 19a to control the vapor inhalation device 10 and to perform operations comprising:

in a first step receiving 52 data from a mobile communication device 20 configured to control the vapor inhalation device 10 in accordance with the APP via the communication unit 17, then determining 54 the amount of first substance a to be released based on the data received 52, and
controlling 56 the release devices 15a, 15b based on the determined amount of first substance a.

According to an illustrative embodiment of the disclosure, the inhalation device can also receive and transmit data related to settings and usage by means of the communication unit.

According to an illustrative embodiment of the disclosure, the inhalation device 10 also comprises at least one of a flow sensor, or a pressure sensor (not illustrated) for providing the communication unit 17 with data about volume of inhalations as determined by the flow, or pressure sensor. In this way, the inhalation device can also monitor an amount of substance delivered to a user.

Advantageously, the inhalation device can control and/or limit amount of substance delivered to help the user limit the consumption of a certain substance such as nicotine.

Advantageously, the inhalation device can also control and/or limit amount of substance delivered such that a user can set a limit on an intake amount of the substance per use or for a certain period by the user.

The inhalation device can also control and/or limit amount of substance delivered to help the user limit the consumption of a certain substance such as nicotine. Advantageously, the inhalation device can also control and/or limit amount of substance delivered such that a user can set a limit on an intake amount of the substance per use or for a certain period by the user.

The purpose of the vapor inhalation device and thereto related apparatus described in this disclosure is to deliver a precise user defined amount of substance a, such as a drug, in particular nicotine. The vapor inhalation is communicating with and controlled by the user's mobile communication device by means of the communication unit via a suitable communication media such as Blue Tooth Low energy (BLE). The user selects nicotine strength in the connected mobile application product APP, which in turn is communicated to the controller 18 of the vapor inhalation device. An intention is to deliver nicotine levels in precise increments from zero % nicotine concentration to high nicotine, say 100% nicotine concentration so that the user can program a tapered nicotine reduction over a specified period of time. For example, the user can program the APP in the mobile communication device that he wishes to taper his nicotine over a time period of 90 days.

The precise nicotine level is achieved by independently heating the separate release devices, in the following referred to as atomizers. Each release device is heated individually by the electric current I provided by the controller, for instance by pulse width modulation

(PWM). The controller sets the PWM duty cycle for each release device in order to achieve the desired mix ratio. For instance, as an example, the first atomizer/reservoir 16a/15a contains a nicotine propylene glycol solution, the second atomizer 16b/15b contains pure propylene glycol and the third atomizer contains propylene glycol and a placebo substance. The first and third atomizers heat to an amount which equals 100% of a predetermined base line. The relationship between the first and the third atomizer can be described as A %+C %=100% baseline. For example, if A heats at 10%, C will heat 90%. The second atomizer always heats at 100%.

The mobile communication device 20 running the APP will transmit information via the communication unit 17 to the controller 18. When a user activates the device, it will transmit information via the communication unit 17 to the APP instead. The transmitted information can include battery charge percentage, number of individual atomizer activations and volume of inhalations as determined by the pressure sensor.

According to an embodiment of the disclosure, there is provided a non-transitory computer-readable medium having instructions stored thereon that, if executed by a computing device in a mobile communications device 20, cause the computing device to control a vapor inhalation device 10 and to perform operations comprising providing 50 data from the mobile communication device 20 configured to control the vapor inhalation device 10.

In this way, for instance an adaptive algorithm monitors how the user uses the device (more specific) and automatically calculates a base line for how much nicotine is administered per inhalation. The user continues using the device as they normally would, and the APP, communicating with the controller, will gradually decrease the nicotine level over a period of time until the user is inhaling nothing but placebo vapor. It is possible that the user will also receive notifications about specific times he should use the device and when he has inhaled an equivalent of a traditional combustible cigarette (combustible cigarette as per user definition of brand and/or strength, i.e. light, gold, ultra light, etc.) and milestones/achievements.

According to an embodiment of the present disclosure, there is provided a cessation algorithm.

An object of the cessation algorithm is to make quitting smoking less difficult by providing highly personalized nicotine dose program. This nicotine dose is based on three elements: data aggregated from all users, baseline values from 3rd party research, and personalized data collected from the user using the vapour inhalation device. Nicotine is optimized to learn a user's nicotine intake habits and then create a unique, customized and dynamically responsive cessation program for each individual user. Below are general characteristics of an embodiment of the algorithm:
The algorithm's function can be divided into two main parts:
1. To control and execute any number of non-dynamic, explicitly programmed instructions for nicotine-cessation or nicotine-reduction “programs”.
   A “program” can be defined as a set of electronic instructions written for the vapour device and/or platform which indicate how the device should dispense nicotine vapour as well as placebo vapour for user consumption over a specific period of time and or a specific number of puffs.
   The program(s) may be stored and accessed electronically.
   The user can select, modify, or create a program by using the App or any other electronic interface.
   An example of a cessation program is where a user may select a program that instructs the vaporizer device to reduce nicotine concentration in a linear trajectory. Whereas a predetermined nicotine-vapour concentration begins at 1800 ppm, whereas following each 24 hour time period the nicotine-vapour concentration is reduced by 18 ppm until a nicotine-vapour concentration of 0 ppm is reached. At the same time, the placebo substance will proportionately increase in concentration in equal parts to the decreased nicotine. As the nicotine is reduced by 18 ppm, the placebo may be increased by 18 ppm over a 24-hour time period until nicotine concentration reaches 0 ppm.
   The store of nicotine cessation programs can be modified by the user using an interface that allows the user to create their own cessation program and save it. The user may specify the duration of the program, the blend of nicotine over the timeframe and even fluctuation within the total timeframe (day parting).
   The cessation programs will be preconfigured by the inhalation device based on 3^{rd} party research and aggregated user data.
2. To control and execute any number of dynamic individually optimized nicotine-cessation or nicotine-reduction “programs” that are executed using any number or combination of computational systems that can learn from data such as, but not limited to:
   Machine-learning, including but not limited to:
      Supervised learning
      Unsupervised learning
      Reinforcement learning
      Semi-supervised learning
      Transduction
   Any kind of machine-learning approach such as:
      Decision tree learning
      Association rule learning
      Artificial neural networks
      Inductive logic programming
      Support vector machines
      Clustering
      Graphical models
      Bayesian networks
      Reinforcement learning
      Representation learning
      Similarity and metric learning
      Sparse Dictionary Learning
   Any kind of statistical analysis such as
      Any type of regression analysis
      Any type of LDA analysis
      Analysis of Variance
      F Tests
      T Tests
      Any type of correlation analysis
   Behavioural-learning
   Any kind of graph analysis
   Artificial intelligence
   Data-mining
   Pattern recognition
   The “program” can be defined as a set of electronic instructions as determined by the previously described computational system which instructs the inhalation device and/or platform to dispense nicotine vapour in prescribed concentration, and a corresponding placebo substance vapour concentration for user inhalation over a specific period of time.
   The user can select a dynamic or non-dynamic program.
The program and the algorithm can be stored and executed on the PCBA inside the inhalation device or the portable mobile communication device used to connect to the inhalation device, (mobile phone, etc.) or on both the inhalation and mobile communication device at the same time.
Data is collected from all users who use the inhalation device, or any device employing the technology platform of this disclosure. The data is stored and accessed from a database. The device-specific data points gathered consist of, but are not limited to:
   User location
   Date and time of each inhalation device activation
   Device battery strength
   Individual cartridge identification number
   Number of inhalation device activations
   Duration of each inhalation device activation
   Volume of vapour inhaled (volume=duration*pressure)
   Spatial proximity to other users
   Device velocity (used to determine the user's activity such as driving, walking, sitting, etc.)
   Make and serial number of mobile communication device being used.
   Successful quit event
On initial activation, the user would also typically be asked to enter personal information such as:
   Age
   Gender
   Weight
   Height
   Education level
   Income
   Number of years as a smoker.
   Social accounts (where some of the above could also be captured)
Optionally users will typically have the opportunity to provide more data
   Users can specify if they crave nicotine at a given moment, they can also be prompted to enter this information
An individual usage profile is generated and continually updated for each individual user. As the user progresses in the cessation program and if the user has selected a dynamic program. The cessation program is personalized based on user-generated dataset (as defined above) generated by using the inhalation device or the platform of this disclosure. The program can be personalized with the object of achieving successful nicotine cessation.
The algorithm employs a diverse array of statistical analysis techniques as detailed above on aggregated inhalation device user data in order to build baseline programs that achieve optimal nicotine cessation. Additionally, patterns in individual nicotine consumption on a per device level is used to personalize the program to achieve a successful outcome. A goal is to optimize and dynamically adjust each individual user's nicotine-reduction program to achieve the highest possible probability of success.
   Cessation optimization is achieved by the algorithm comparing individual user data to the larger set of all user data to search for positive correlation of nicotine reduction patterns with the percentage of successful user cessation attempts.

The canonical cessation program derived from aggregate inhalation device user data would employ linear regression and statistical tests. A successful cessation event would be defined as the dependent variable, and the independent variables would be defined as the user specific information such as the users' age, weight, and their usage patterns, e.g., number of puffs per day, morning intake vs evening intake (day part). The algorithm would run logistic regression with a matrix of dependent variables and return the “r” values of the variables. The algorithm would also return “p” values to ensure a degree of certainty that the results were within an acceptable confidence interval. The variables with the highest “r” values would be used to build the program. This type of multivariate analysis is computationally intensive and would occur on a weekly basis.

According to an aspect of this disclosure, the canonical personalized cessation program would use the same technique as the overall cessation program but on a reduced set of independent variables. The personalized program could also use ‘craving’ as a dependent variable and optimize to eliminate personal craving events.

An example of a machine-learning cessation program is such that the user begins using the inhalation device according to their personal need for nicotine craving mitigation. The user continues to use the inhalation device over a period of time until the algorithm reaches a point where it determines the user's “baseline” behaviour and daily nicotine requirement. Upon this determination, the algorithm initiates an automatic nicotine reduction program that continuously adjusts nicotine concentration levels according to user behaviour and device usage (as determined by factors listed in “data points” above). Nicotine concentration may be decreased or increased over any given time period, however, the long term trajectory for the user's nicotine consumption trends negative until reaching full nicotine cessation. The process as described above is entirely automatic as controlled by the machine-learning algorithm so that device requires zero user input.

According to an embodiment of the present disclosure, the cartridge can be equipped with a Near Field Communication (NFC) RFID chip. The NFC chip will identify each cartridge by assigning the cartridge a unique identifier such as Unique identifying Cartridge Number. The cartridge identifiers can be stored in a data base (not shown) and when the user inserts a cartridge, this will be notified in the data base. Typically, the purpose of the NFC chip is two-fold, such as to keep track of each cartridge's use. For example, if the user inserts a partially used cartridge, the vapor inhalation device will typically communicate with the data base to understand exactly how much fluid is remaining in the reservoir(s). This will allow to seamlessly continuing the user's program while displaying accurate information about fluid levels. This will also serve as an anti-piracy and anti-tampering measure. If the cartridge does not match the database, the cartridge will simply not work. In other applications, for instance if a doctor has prescribed a controlled substance to be inhaled using the inventive vapor inhalation device, this allows for the doctor to activate or deactivate an individual cartridge through a database interface.

In other embodiments, one or any number of cartridges can be used.

General Outline of Unique Identifying Cartridge Number (UICN)

A purpose for the UICN is, but not limited to:
Asset and product tracking
Inventory systems
Cartridge Access control (For example, a physician or other medical professional could render a cartridge inoperable wirelessly and via distance by electronic information transmission of any kind)
Cartridge Authentication & Anti-piracy
Automatic cartridge identification and cartridge data capture
Quality Control and Verification
Distributed Sensor Networks

The UICN can be described as such:
Each individual cartridge for use in the inhalation device will be assigned a unique identifying cartridge number (UICN).
All UICNs will be electronically stored in a central database for later retrieval.
When the cartridge is inserted into the vaporizer device, the UICN will be read and matched against a central database of UICNs.
The UICN can be stored and accessed through any number of information transmission systems such as, but not limited to:
   Integrated Circuit or Microchip transponder
   ASIC-based RFID tags
   Frequency signature type RFIDs
   Time domain reflectometry type RFIDs
   Chipless RFID tags such as, but not limited to:
      Chemical-based RFID tags
      Radiation-based RFID tags
      Dielectric ink
      Nanometric ink (magnetism-based)
      Programmable magnetic resonance (Acousto-magnetic devices)
      Flying Null technology
      Surface acoustic wave based RFIDs
      Capacitively tuned split microstrip resonators
      Nano-resonant structures such as Tapemark's chipless ID
   Any kind of optical machine-readable representation of data
   Any kind of device that employs chemical, magnetic materials, or resonant circuits to attenuate or absorb radiation of a particular frequency in order to transmit information.

1. A vapor inhalation device comprising:
a housing adapted to enable a user to inhale vapor, simulating the effects of smoking, the housing being adapted to receive a cartridge for containing liquids;
the cartridge comprising or connected to a first release device arranged to vaporize and release a first liquid comprising a first substance from a first reservoir into the housing; and to a second release device arranged to vaporize and release a second liquid from a second reservoir, wherein the housing is adapted to mix the vaporized first and second liquids such that the user can inhale vapor containing the first substance of a particular amount;
a communication unit configured to receive and transmit data for controlling the inhalation device;
a controller configured to communicate with the communication unit and to receive data from the communication unit; and to
determine an amount of first substance to be released based on the data; and
control the first and second release devices based on the determined amount of the first substance.

2. The inhalation device according to claim 1, wherein the communication unit is configured to communicate with and be controlled by a user's mobile communication device having an application program product, APP, configured to control the inhalation device.

3. The inhalation device according to claim 2, wherein the controller is configured to control the first and second release devices to deliver the first substance in precise increments, which can be set by the user, in an amount of 0 to 100% concentration of the first substance.

4. The inhalation device according to claim 1, where and the first and second release devices comprises at least one of a heating element, a vibration generator an ultrasonic transducer, a piezoelectric transducer, and an atomizer.

5. The inhalation device according to claim 4, wherein the first and second release devices comprise atomizers, each comprising a vaporizing coil that can be heated by electric current provided by a battery provided in the housing.

6. The inhalation device according to claim 5, further comprising a third release device, wherein the first reservoir contains the first liquid being a solvent and the first substance, the second reservoir contains the first liquid only and the third reservoir contains the first liquid and a placebo substance.

7. The inhalation device according to claim 6, wherein the release devices and/or the reservoirs are arranged in parallel to each other.

8. The inhalation device according to claim 6, wherein the release devices and/or the reservoirs are arranged in series to each other.

9. The inhalation device according to claim 1, further comprising at least one of a flow sensor, or a pressure sensor for providing the communication unit with data about volume of inhalations as determined by the flow, or pressure sensor, which communication unit is configured to transmit the data to the mobile communication device.

10. The inhalation device according to claim 9, wherein the communication unit is configured to transmit data including at least one of battery charge percentage, or number of individual atomizer activations.

11. A cartridge containing liquids for the vapor inhalation device according to claim 1, the cartridge comprising or connectable to a first release device arranged to vaporize and release a first liquid comprising a first substance from a first reservoir into the housing; and to a second release device arranged to vaporize and release a second liquid from a second reservoir.

12. The cartridge according to claim 11, further comprising or being connectable to a third release device arranged to vaporize and release a third liquid from a third reservoir of the cartridge, wherein the first reservoir contains the first liquid being a solvent and the first substance, the second reservoir contains the first liquid only and the third reservoir contains the first liquid and a placebo substance.

13. The cartridge according to claim 1, wherein the first substance comprises a drug.

14. The cartridge according to claim 13, wherein the drug comprises nicotine.

15. A controller comprising a computing device and non-transitory computer-readable medium having instructions stored thereon that, if executed by the computing device, cause the computing device to control a vapor inhalation device and to perform operations comprising:
receiving data from a mobile communication device such as a smart-phone having an application program product, APP, configured to control a vapor inhalation device, comprising a housing adapted to enable a user to inhale vapor, simulating the effects of smoking, the housing being adapted to receive a cartridge for containing liquids;
the cartridge comprising a first release device arranged to vaporize and release a first liquid comprising a first substance into the housing; and a second release device arranged to vaporize and release a second liquid, wherein the housing is adapted to mix the vaporized first and second liquids such that the user can inhale vapor containing the first substance of a particular amount;
a communication unit configured to receive and transmit data for controlling the inhalation device;
the controller being configured to communicate with the communication unit and to receive data from the communication unit and to control the first and second release devices;
determining the amount of first substance to be released based on the data received; and
controlling the first and second release devices based on the determined amount of first substance.

16. A non-transitory computer-readable medium having instructions stored thereon that, if executed by a computing device in a mobile communications device, cause the computing device to control a vapor inhalation device and to perform operations comprising:
providing data from the mobile communication device configured to control the vapor inhalation device according to claim 1.
